SEQUENCE LISTING (69 sequences)

SEQ ID NO: 1 - protein, 243 aa, Homo sapiens
  MISC_FEATURE (8)..(8): Xaa is a tryptophan residue or an oxidant resistant residue
DEPPQSQXDRVKDLATVYVDVLKDSGRDYVSQFEGSALGKQLNLKLLDNXDSVTSTFSKLREQLGPVTQEFXDNLEKETEGLRQEMSKDLEEVKAKVQPYLDDFQKKXQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 267 aa, Homo sapiens
  MISC_FEATURE (22)..(22): Xaa is a tryptophan residue or an oxidant resistant residue
MKAAVLTLAVLFLTGSQARHFXQQDEPPQSPXDRVKDLATVYVDVLKDSGRDYVSQFEGSALGKQLNLKLLDNXDSVTSTFSKLREQLGPVTQEFXDNLEKETEGLRQEMSKDLEEVKAKVQPYLDDFQKKXQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 267 aa, Homo sapiens
  MISC_FEATURE (22)..(22): Xaa is a tryptophan residue or an oxidant resistant residue
MKAAVLTLAVLFLTGSQARHFXQQDEPPQSPXDRVKDLATVYVDVLKDSGRDYVSQFEGSALGKQLNLKLLDNXDSVTSTFSKLREQLGPVTQEFXDNLEKETEGLRQEMSKDLEEVKAKVQPYLDDFQKKXQEEMELYRQKVEPLRAELQEGARQKLHELQEKLSPLGEEMRDCARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 18 aa, Artificial Sequence
  Synthetic
DXLKAFYDKVAEKLKEAF

SEQ ID NO: 5 - protein, 18 aa, Artificial Sequence
  Synthetic
DXFKAFYDKVAEKLKEAF

SEQ ID NO: 6 - protein, 18 aa, Artificial Sequence
  Synthetic
DXLKAFYDKVAEKFKEAF

SEQ ID NO: 7 - protein, 18 aa, Artificial Sequence
  Synthetic
DXFKAFYDKVAEKFKEAF

SEQ ID NO: 8 - protein, 18 aa, Artificial Sequence
  Synthetic
DXLKAFYDKVFEKFKEFF

SEQ ID NO: 9 - protein, 18 aa, Artificial Sequence
  Synthetic
DXLKAFYDKFFEKFKEFF

SEQ ID NO: 10 - protein, 18 aa, Artificial Sequence
  Synthetic
DXFKAFYDKFFEKFKEFF

SEQ ID NO: 11 - protein, 18 aa, Artificial Sequence
  Synthetic
DXLKAFYDKVFEKFKEAF

SEQ ID NO: 12 - protein, 18 aa, Artificial Sequence
  Synthetic
DXLKAFYDKVFEKFKEAF

SEQ ID NO: 13 - protein, 18 aa, Artificial Sequence
  Synthetic
DXLKAFYDKVFEKLKEFF

SEQ ID NO: 14 - protein, 18 aa, Artificial Sequence
  Synthetic
DXLKAFYDKVAEKFKEFF

SEQ ID NO: 15 - protein, 18 aa, Artificial Sequence
  Synthetic
DXLKAFYDKVFEKFKEFF

SEQ ID NO: 16 - protein, 18 aa, Artificial Sequence
  Synthetic
EXLKLFYEKVLEKFKEAF

SEQ ID NO: 17 - protein, 18 aa, Artificial Sequence
  Synthetic
EXLKAFYDKVAEKFKEAF

SEQ ID NO: 18 - protein, 18 aa, Artificial Sequence
  Synthetic
EXLKAFYDKVAEKLKEFF

SEQ ID NO: 19 - protein, 18 aa, Artificial Sequence
  Synthetic
EXLKAFYDKVFEKFKEAF

SEQ ID NO: 20 - protein, 18 aa, Artificial Sequence
  Synthetic
EXLKAFYDKVFEKLKEFF

SEQ ID NO: 21 - protein, 18 aa, Artificial Sequence
  Synthetic
EXLKAFYDKVAEKFKEFF

SEQ ID NO: 22 - protein, 18 aa, Artificial Sequence
  Synthetic
EXLKAFYDKVFEKFKEFF

SEQ ID NO: 23 - protein, 18 aa, Artificial Sequence
  Synthetic
DXLKALYDKVAEKLKEAL

SEQ ID NO: 24 - protein, 18 aa, Artificial Sequence
  Synthetic
DXFKAFYEKVAEKLKEFF

SEQ ID NO: 25 - protein, 18 aa, Artificial Sequence
  Synthetic
DXFKAFYEKFFEKFKEFF

SEQ ID NO: 26 - protein, 18 aa, Artificial Sequence
  Synthetic
EXLKALYEKVAEKLKEAL

SEQ ID NO: 27 - protein, 18 aa, Artificial Sequence
  Synthetic
EXLKAFYEKVAEKLKEAF

SEQ ID NO: 28 - protein, 18 aa, Artificial Sequence
  Synthetic
EXFKAFYEKVAEKLKEFF

SEQ ID NO: 29 - protein, 18 aa, Artificial Sequence
  Synthetic
EXLKAFYEKVFEKFKEFF

SEQ ID NO: 30 - protein, 18 aa, Artificial Sequence
  Synthetic
EXLKAFYEKFFEKFKEFF

SEQ ID NO: 31 - protein, 18 aa, Artificial Sequence
  Synthetic
EXFKAFYEKFFEKFKEFF

SEQ ID NO: 32 - protein, 18 aa, Artificial Sequence
  Synthetic
DFLKAXYDKVAEKLKEAX

SEQ ID NO: 33 - protein, 18 aa, Artificial Sequence
  Synthetic
EFLKAXYEKVAEKLKEAX

SEQ ID NO: 34 - protein, 18 aa, Artificial Sequence
  Synthetic
DFXKAXYDKVAEKLKEXX

SEQ ID NO: 35 - protein, 18 aa, Artificial Sequence
  Synthetic
EFXKAXYEKVAEKLKEXX

SEQ ID NO: 36 - protein, 18 aa, Artificial Sequence
  Synthetic
DKLKAFYDKVFEXAKEAF

SEQ ID NO: 37 - protein, 18 aa, Artificial Sequence
  Synthetic
DKXKAVYDKFAEAFKEFL

SEQ ID NO: 38 - protein, 18 aa, Artificial Sequence
  Synthetic
EKLKAFYEKVFEXAKEAF

SEQ ID NO: 39 - protein, 18 aa, Artificial Sequence
  Synthetic
EKXKAVYEKFAEAFKEFL

SEQ ID NO: 40 - protein, 18 aa, Artificial Sequence
  Synthetic
DXLKAFVDKFAEKFKEAY

SEQ ID NO: 41 - protein, 18 aa, Artificial Sequence
  Synthetic
EKXKAVYEKFAEAFKEFL

SEQ ID NO: 42 - protein, 18 aa, Artificial Sequence
  Synthetic
DXLKAFVYDKVFKLKEFF

SEQ ID NO: 43 - protein, 18 aa, Artificial Sequence
  Synthetic
EXLKAFVYEKVFKLKEFF

SEQ ID NO: 44 - protein, 18 aa, Artificial Sequence
  Synthetic
DXLRAFYDKVAEKLKEAF

SEQ ID NO: 45 - protein, 18 aa, Artificial Sequence
  Synthetic
EXLRAFYEKVAEKLKEAF

SEQ ID NO: 46 - protein, 18 aa, Artificial Sequence
  Synthetic
DXLKAFYDRVAEKLKEAF

SEQ ID NO: 47 - protein, 18 aa, Artificial Sequence
  Synthetic
EXLKAFYERVAEKLKEAF

SEQ ID NO: 48 - protein, 18 aa, Artificial Sequence
  Synthetic
DXLKAFYDKVAERLKEAF

SEQ ID NO: 49 - protein, 18 aa, Artificial Sequence
  Synthetic
EXLKAFYEKVAERLKEAF

SEQ ID NO: 50 - protein, 18 aa, Artificial Sequence
  Synthetic
DXLKAFYDKVAEKLREAF

SEQ ID NO: 51 - protein, 18 aa, Artificial Sequence
  Synthetic
EXLKAFYEKVAEKLREAF

SEQ ID NO: 52 - protein, 18 aa, Artificial Sequence
  Synthetic
DXLKAFYDRVAERLKEAF

SEQ ID NO: 53 - protein, 18 aa, Artificial Sequence
  Synthetic
EXLKAFYERVAERLKEAF

SEQ ID NO: 54 - protein, 18 aa, Artificial Sequence
  Synthetic
DXLRAFYDKVAEKLREAF

SEQ ID NO: 55 - protein, 18 aa, Artificial Sequence
  Synthetic
EXLRAFYEKVAEKLREAF

SEQ ID NO: 56 - protein, 18 aa, Artificial Sequence
  Synthetic
DXLRAFYDRVAEKLKEAF

SEQ ID NO: 57 - protein, 17 aa, Artificial Sequence
  Synthetic
XLRAFYERVAEKLKEAF

SEQ ID NO: 58 - protein, 18 aa, Artificial Sequence
  Synthetic
DXLKAFYDKVAERLREAF

SEQ ID NO: 59 - protein, 18 aa, Artificial Sequence
  Synthetic
EXLKAFYEKVAERLREAF

SEQ ID NO: 60 - protein, 18 aa, Artificial Sequence
  Synthetic
DXLRAFYDKVAERLKEAF

SEQ ID NO: 61 - protein, 18 aa, Artificial Sequence
  Synthetic
EXLRAFYEKVAERLKEAF

SEQ ID NO: 62 - protein, 37 aa, Artificial Sequence
  Synthetic
DXLKAFYDKVAEKLKEAFPDXLKAFYDKVAEKLKEAF

SEQ ID NO: 63 - protein, 37 aa, Artificial Sequence
  Synthetic
DXLKAFYDKVAEKLKEFFPDXLKAFYDKVAEKLKEFF

SEQ ID NO: 64 - protein, 37 aa, Artificial Sequence
  Synthetic
DXFKAFYDKVAEKLKEAFPDXFKAFYDKVAEKLKEAF

SEQ ID NO: 65 - protein, 37 aa, Artificial Sequence
  Synthetic
DKLKAFYDKVFEXAKEAFPDKLKAFYDKVFEXLKEAF

SEQ ID NO: 66 - protein, 37 aa, Artificial Sequence
  Synthetic
DKXKAVYDKFAEAFKEFLPDKXKAVYDKFAEAFKEFL

SEQ ID NO: 67 - protein, 37 aa, Artificial Sequence
  Synthetic
DXFKAFYDKVAEKFKEAFPDXFKAFYDKVAEKFKEAF

SEQ ID NO: 68 - protein, 37 aa, Artificial Sequence
  Synthetic
DXLKAFVYDKVFKLKEFFPDXLKAFVYDKVFKLKEFF

SEQ ID NO: 69 - protein, 37 aa, Artificial Sequence
  Synthetic
DXLKAFYDKFAEKFKEFFPDXLKAFYDKFAEKFKEFF

(19) United States
(12) Patent Application Publication
(10) Pub. No.: US 2012/0189612 A1
(43) Pub. Date: Jul. 26, 2012

(54) COMPOSITIONS AND METHODS FOR TREATING CANCER WHILE PREVENTING OR REDUCING CARDIOTOXICITY AND/OR CARDIOMYOPATHY

(75) Inventor: Stanley L. Hazen, Pepper Pike, OH (US)

(73) Assignee: THE CLEVELAND CLINIC FOUNDATION, Cleveland, OH (US)

(21) Appl. No.: 13/357,668

(22) Filed: Jan. 25, 2012

Related U.S. Application Data
(60) Provisional application No. 61/436,036, filed on Jan. 25, 2011.
(60) Provisional application No. 61/468,292, filed on Mar. 28, 2011.

(51) Int. Cl. A61K 38/17; A61P 9/00; A61K 39/395; A61P 35/00; A61K 31/7105; A61K 31/455

(52) U.S. Cl. 424/130.1; 514/19.3; 514/44 R; 514/356

(57) ABSTRACT

The present invention provides methods for treating cardiotoxicity, cardiomyopathy, and/or cancer in a subject, as well as related compositions and kits, that employ a therapeutic agent, or a nucleic acid sequence encoding a therapeutic agent, selected from apolipoprotein A-1 (ApoA1), an ApoA1 mimetic, an agent that increases expression of ApoA1, or a binding agent specific for oxidized ApoA1, where the therapeutic agent is effective in preventing or reducing the level of cardiotoxicity and/or cardiomyopathy induced by the chemotherapeutic.

### CROSS-REFERENCE TO RELATED APPLICATIONS

The present application claims the benefit of U.S. Provisional Patent Application Ser. No. 61/436,036, filed Jan. 25, 2011, and U.S. Provisional Patent Application Ser. No. 61/468,292, filed Mar. 28, 2011, each of which is incorporated by reference in its entirety.

### FIELD OF THE INVENTION

The present invention relates to methods for treating cardiotoxicity, cardiomyopathy, and/or cancer in a subject, as well as related compositions and kits, that employ a therapeutic agent, or a nucleic acid sequence encoding a therapeutic agent, selected from apolipoprotein A-1 (ApoA1), an ApoA1 mimetic, an agent that increases expression of ApoA1, or a binding agent specific for oxidized ApoA1, where the therapeutic agent is effective in preventing or reducing the level of cardiotoxicity and/or cardiomyopathy induced by the chemotherapeutic.

### BACKGROUND OF THE INVENTION

Circulating cholesterol is carried by plasma lipoproteins. Lipoproteins are particles of lipid and protein that transport lipids in the blood. Low-density lipoproteins (LDL) and high-density lipoproteins (HDL) are the major cholesterol carriers. LDL is believed to be responsible for the delivery of cholesterol from the liver to extrahepatic tissues in the body.

The term “reverse cholesterol transport” (RCT) describes the transport of cholesterol from extrahepatic tissues to the liver where it is catabolized and eliminated. It is believed that plasma HDL particles play a major role in the reverse transport process, acting as scavengers of tissue cholesterol. RCT consists mainly of three steps: (a) cholesterol efflux, the initial removal of cholesterol from various pools of peripheral cells; (b) cholesterol esterification by the action of lecithin:cholesterol acyltransferase (LCAT), preventing a re-entry of effluxed cholesterol into cells; and (c) uptake/delivery of HDL cholesteryl ester to liver cells.

High levels of HDL and apolipoprotien A-1 (ApoA1), the major HDL protein, have long been associated with decreased risk for cardiovascular disease. ApoA1 is a single polypeptide chain with 243 amino acid residues of known primary amino acid sequence (Brewer et al., (1978) Biochem. Biophys. Res. Commun. 80:623-630). ApoA1 acts as an acceptor of cellular cholesterol in the reverse cholesterol transport by mediating cholesterol efflux from cells. Each HDL particle contains at least one copy (and usually two to four copies) of ApoA1. ApoA1 is synthesized in humans in the form of a preproapolipoprotein of 267 residues by the liver and small intestine which is secreted as a proprotein that is rapidly cleaved by the action of a calcium-dependent protease to generate a mature 243 amino acid polypeptide and secreted into the plasma. ApoA1 has been postulated to possess eight tandem repeating 22 mer sequences and two 11 mer sequences, most of which have the potential to form class A amphipathic helical structures (Segrest et al. (1974) FEBS Lett. 38:247-253). Characteristics of the class A amphipathic helix including the presence of positively charged residues at the polar-nonpolar interface and negatively charged residues at the center of the polar face (Segrest et al. (1974) FEBS Lett. 38:247-253; Segrest et al. (1990) Proteins: Structure, Function, and Genetics 8:103-117).

ApoA1 forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL₃ and HDL₂). Most HDL in the circulating population contain both ApoA1 and ApoAII (the second major HDL protein) and are referred to as the A1/AII-HDL fraction of HDL. However, the fraction of HDL containing only ApoA1 (referred to herein as A1-HDL fraction) appear to be more effective in RCT. Certain epidemiologic studies support the hypothesis that the A1-HDL fraction is anti-atherogenic. (Parra et al., 1992, Arterioscler. Thromb. 12:701-707; Decossin et al., 1997, Eur. J. Clin. Invest. 27:299-307).

### SUMMARY OF THE INVENTION

The present invention provides methods for treating cardiotoxicity, cardiomyopathy, and/or cancer in a subject, as well as related compositions and kits, that employ a therapeutic agent, or a nucleic acid sequence encoding a therapeutic agent, selected from apolipoprotein A-1 (ApoA1), an ApoA1 mimetic, an agent that increases expression of ApoA1, or a binding agent specific for oxidized ApoA1, where the therapeutic agent is effective in preventing or reducing the level of cardiotoxicity and/or cardiomyopathy induced by the chemotherapeutic.

In some embodiments, the present invention provides methods of treating cancer in a subject comprising: administering to a subject: a) a chemotherapeutic agent, wherein the chemotherapeutic agent induces at least some level of cardiotoxicity or cardiomyopathy in the subject when administered at a therapeutic level, and b) a composition comprising a therapeutic agent and/or a nucleic acid sequence encoding the therapeutic agent, wherein the therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in the subject, wherein said composition is effective in preventing or reducing the level of cardiotoxicity and/or cardiomyopathy induced by the chemotherapeutic agent, and wherein the administering is effective to prevent, suppress, and/or inhibit cancer cell growth in the subject.

In particular embodiments, the chemotherapeutic agent is administered with the composition to the subject at a dosage level that causes about the same level of cardiotoxicity and/or cardiomyopathy induced when the chemotherapeutic agent is administered without the composition to the subject.

In some embodiments, the present invention provides methods of treating cardiotoxicity or cardiomyopathy comprising: administering, to a subject with cardiotoxicity or cardiomyopathy, a composition comprising a therapeutic agent and/or a nucleic acid sequence encoding the therapeutic agent, wherein the therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in said subject, wherein the administering is effective in preventing or reducing the level of cardiotoxicity and/or cardiomyopathy in the subject. In particular embodiments, the cardiotoxicity or cardiomyopathy is induced by a chemotherapeutic agent previously taken by the subject.

In certain embodiments, the therapeutic agent comprises a high-density lipoprotein (HDL). In other embodiments, the therapeutic agent comprises preproapoliprotein (preproApoA1). In other embodiments, the composition is injected and/or infused into the subject. In additional embodiments, the nucleic acid sequence comprises an ApoA1 mRNA sequence. In further embodiments, the nucleic acid sequence comprises an ApoA1 mimetic mRNA sequence. In additional embodiments, the nucleic acid sequence comprises a DNA sequence encoding the therapeutic agent. In other embodiments, the nucleic acid sequence comprises an expression vector.

In some embodiments, the chemotherapeutic agent is selected from the group consisting of: an anthracycline, an anthraquinones, doxorubicin, daunorubicin, epirubicin, idarubicin, a high-dose alkylating agents, cisplatin, cyclophosphamide, ifosfamide, mitomycin, an anti-metabolites, 5-fluorouracil, cytarabine, Paclitaxel, Trastuzumab, Alemtuzumab, Campath, a VSP inhibitors/Tyrosine kinase inhibitor, imatinib, bevacizumab, sorafenib, sunitinib, pazopanib, dasatinib, nilotinib, and mitoxantrone.

In certain embodiments, the therapeutic agent comprises a small molecule compound. In other embodiments, the therapeutic agent comprises a nucleic acid sequence encoding the HNF-4 gene or active portion thereof. In further embodiments, the therapeutic agent comprises a statin that is able to cause an increased expression of HDL or ABCA1. In other embodiments, the statin comprises Atorvastatin. In other embodiments, endurance exercise training is prescribed to raise HDL and therefore increase expression of ApoA1. In other embodiments, the therapeutic agent is niacin. In further embodiments, the therapeutic agent is a fibrate that is able to cause increased expression of HDL. In other embodiments, the fibrate is selected from the group consisting of: Fenofibrate, bezafibrate, gemfibrozil, and LY518674.

In some embodiments, the present invention provides pharmaceutical composition for treating cancer in a subject comprising: a) a chemotherapeutic agent, wherein the chemotherapeutic agent induces at least some level of cardiotoxicity or cardiomyopathy in the subject when administered at a therapeutic level, and b) a therapeutically effective amount of a therapeutic agent and/or a nucleic acid sequence encoding said therapeutic agent, wherein the therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in the subject.

In other embodiments, the therapeutic agent comprises a high-density lipoprotein. In additional embodiments, the therapeutic agent comprises preproapoliprotein (preproApoA1). In some embodiments, the nucleic acid sequence comprises an ApoA1 mRNA sequence. In particular embodiments, the nucleic acid sequence comprises an ApoA1 mimetic mRNA sequence. In further embodiments, the nucleic acid sequence comprises a DNA sequence encoding the therapeutic agent. In other embodiments, the nucleic acid sequence comprises an expression vector.

In particular embodiments, the binding agent comprises an antibody or antigen-binging portion thereof. In other embodiments, the antibody is the 10C5.2 monoclonal antibody, or an antigen-binding portion of the 10C5.2 monoclonal antibody. In other embodiments, the binding agent comprises at least one variable region, or at least one CDR, from the 10C5.2 monoclonal antibody. In some embodiments, the antibody is the 10G1.5 monoclonal antibody, or an antigen-binding portion of the 10G1.5 monoclonal antibody. In further embodiments, the binding agent comprises at least one variable region, or at least one CDR, from the 10G1.5 monoclonal antibody. In additional embodiments, the antibody is the 4G11.2 monoclonal antibody, or an antigen-binding portion of the 4G11.2 monoclonal antibody. In other embodiments, the binding agent comprises at least one variable region, or at least one CDR, from the 4G11.2 monoclonal antibody.

In certain embodiments, the present invention provides methods of treating or preventing cancer is a subject comprising: administering to a subject: a) a chemotherapeutic that induces at least some level of cardiotoxicity or cardiomyopathy, and b) an HDL mimetic peptide, wherein said composition is effective in preventing or reducing the level of cardiotoxicity and/or cardiomyopathy induced by the chemotherapeutic agent, and wherein the administering is effective to prevent, suppress, and/or inhibit cancer cell growth in the subject. In some embodiments, the HDL mimetic peptide is LSI-518P from Lipid Sciences, Inc. In further embodiments, the HDL mimetic peptide is mimetic peptide 4F (see Vakili et al., Adv Exp Med Biol. 2010; 660:167-72, herein incorporated by reference). In certain embodiments, the HDL mimetic peptide is ATI-5261 (see, Bielicki et al., J Lipid Res. 2010 June; 51(6):1496-503, herein incorporated by reference). In other embodiments, the HDL mimetic is as shown in D'Souza et al., Circulation Research. 2010; 107:217, herein incorporated by reference.

In an aspect of the invention, the compositions are administered to a subject with or at risk of cancers including but not limited to malignant melanoma, lung, breast, colon, brain, kidney, bladder, prostate, pancreatic, oral, head and neck, sarcomas, lymphomas (B-cell and T-cell) and adrenal cancer.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “polynucleotide sequence,” “nucleic acid sequence,” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e. deletion, substitution, addition, or alteration) in a wild type polynucleotide sequence or any change in a wild type protein. The term “variant” is used interchangeably with “mutant.” Although it is often assumed that a change in the genetic material results in a change of thee function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

As used herein, the term “nucleic acid” refers to poly nucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors.”

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the poly nucleotide sequence under the control of the expression control sequence, and production of the desired polypeptide encoded by the polynucleotide sequence.

“Homology” and “identity” are used synonymously throughout and refer to sequence similarity between two peptides or between tow nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

A “chimeric protein” or “fusion protein” is a fusion of a first amino acid sequence encoding a polypeptide with a second amino acid sequence defining a domain (e.g., polypeptide portion) foreign to and not substantially homologous with any domain of the first polypeptide. A chimeric protein may present a foreign domain which is found (albeit in a different protein) in an organism which also expresses the first protein, or it may be an “interspecies,” “intergenic,” etc. fusion of protein structures expressed by different kind of organisms.

The phrases “parenteral administration” and “administered paternterally” as used herein means modes of administration other than enteral and topical administration, usually by injection, and includes, without limitation, intravenous, intramuscular, intraaterial, intrathecal, intraventricular, intracapsular, intraorbital, intracarida, intradermal, intraperitoneal, transtracheal, subcutaneous, subcuticular, intraarticular, subcapsular, subarachnoid, intraspinal and transternal injection and infusion. The compositions of the present invention may be administered by parenteral administration.

The phrases “systemic administration,” administered systemically,” “peripheral administration,” and “administered peripherally” as used herein mean the administration of a compound, drug or other material other than directly into the central nervous system, such that it enters the animal's system and, thus, is subject to metabolism and other like processes, for example, subcutaneous administration. The compositions of the present invention may be administered by systemic administration.

### DETAILED DESCRIPTION

The present invention provides methods for treating cardiotoxicity, cardiomyopathy, and/or cancer in a subject, as well as related compositions and kits, that employ a therapeutic agent, or a nucleic acid sequence encoding a therapeutic agent, selected from apolipoprotein A-1 (ApoA1), an ApoA1 mimetic, an agent that increases expression of ApoA1, or a binding agent specific for oxidized ApoA1, where the therapeutic agent is effective in preventing or reducing the level of cardiotoxicity and/or cardiomyopathy induced by the chemotherapeutic.

### I. Combination Therapy

The present invention provides methods of administering both a chemotherapeutic agent (e.g., that induces cardiotoxicity and/or cardiomyopathy) and an ApoA1-related therapeutic agent (e.g., an agent that comprises ApoA1 (or variant thereof), a nucleic acid encoding ApoA1 (or variant thereof), a binding agent specific for oxidized ApoA1, and molecule that induce expression of ApoA1). The chemotherapeutic agent and ApoA1-related therapeutic agent maybe administered together (e.g., in a single composition) or in any temporal order such that the ApoA1-related therapeutic agent helps prevent or reduce the cardiotoxicity and/or cardiomyopathy induced by the chemotherapeutic agent (e.g., administered within 1 minutes . . . 5 minutes . . . 10 minutes . . . 1 hour . . . 2 hours . . . three hours . . . 12 hours . . . or more of each other).

In certain embodiments, this combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by an appropriate route including, but not limited to, oral routes, intravenous routes, intramuscular routes, and direct absorption through mucous membrane tissues. The therapeutic agents can be administered by the same route or by different routes. The sequence in which the therapeutic agents are administered is not narrowly critical.

Combination therapy also can embrace the administration of the therapeutic agents as described above in further combination with other biologically active ingredients (such as, but not limited to, a third and different therapeutic agent) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporarily removed from the administration of the therapeutic agents, perhaps by days or even weeks.

### II. HDL, ApoA1, and Variants Thereof

The present invention is not limited by the methods or compositions used to increase HDL, ApoA1, and related variants in a subject to prevent or reduce the cardiotoxicity or cardiomyopathy induced by a chemotherapeutic agent. Exemplary methods and compositions are described below.

High density lipoprotein (HDL) administered to the subject in accordance with the present invention can include a lipid-protein complex or derivative thereof which when isolated from plasma by ultracentrifugation is found in the density range of d=1.063 to d=1.21. In one embodiment of the invention, HDL can be isolated from human plasma and administered to a subject to protect against a wide variety of cancers, such as malignant melanoma, lung, breast, colon, brain, kidney, bladder, prostate, pancreatic, oral, head and neck, sarcomas, lymphomas (B-cell and T-cell) and adrenal cancer development; as well as to slow development of tumors, tumor metastasis and lengthens survival times. Preferably, the HDL or derivative thereof is a peptide or protein derivative of the sequence of ApoA1, or a peptide or protein mimetic functionally homologous to the active portions of ApoA1.

In one embodiment, the HDL used in accordance with the present invention is reconstituted HDL. The term “reconstituted HDL” means HDL composed of a lipid or lipids in association with at least one of the apolipoproteins of HDL. The components may be derived, for example, from blood, or produced by recombinant technology. For example, nascent HDL can be prepared from ApoA1 isolated from human plasma combined with a wide-range of phospholipids, such as from 5-100:1 molar ratio of phospholipids to apoA1, or 100:1 molar ratio of phospholipids to apoA1, or lipid free or lipid poor ApoA1 (e.g., ratio of 5:1), or ApoA1 with a high lipid content; and reconstituted into nascent HDL particle that can be administered to a subject to treat or prevent cancer or used to treat cardiotoxicity or cardiomyopathy in a subject. In another embodiment, HDL can include monomeric and multimeric HDL peptide mimetics that can function to allow HDL remodeling leading to greater anti-inflammatory activity and cholesterol efflux activity.

ApoA1 administered in accordance with the present invention can include a full-length human ApoA1 peptide or to a fragment or domain thereof comprising a class A amphipathic helix. In some embodiments, ApoA1 can be combined with a wide-range of phospholipids, such as from 5-100:1 molar ratio of phospholipids to ApoA1, reconstituted into a nascent HDL particle, and administered to a subject to treat or prevent cancer. The ApoA1 reconstituted into a HDL particle and administered to a subject can be protective against a wide variety of cancers including, malignant melanoma, lung, breast, colon, brain, kidney, bladder, prostate, pancreatic, oral, head and neck, sarcomas, lymphomas (B-cell and T-cell) and adrenal cancer development, slow development of tumors, and lengthens survival times of the subject.

In other embodiments, ApoA1 can be administered in combination with a wide-range of phospholipids and cholesterol, such as from 5-100:1 molar ratio of phospholipids:cholesterol:ApoA1 and reconstituted into a nascent HDL cholesterol particle. The ApoA1 reconstituted into a HDL cholesterol particle and administered to a subject is protective against a wide variety of cancers including, malignant melanoma, lung, breast, colon, brain, kidney, bladder, prostate, pancreatic, oral, head and neck, sarcomas, lymphomas (B-cell and T-cell) and adrenal cancer development, slow development of tumors, and lengthens survival times of the subject.

The ApoA1 administered to a subject to treat cancer can also include ApoA1 mimetics. The terms “mimetics of ApoA1” or “known mimetics of ApoA1,” or “ApoA1 mimetics,” refer to mimetics of ApoA1 that can be identified or derived from any reference and that have ApoA1 behavior. These include mimetics of ApoA1 identified in U.S. and foreign patents and publications.

ApoA1 mimetics can include natural variants of ApoA1 that are known in the art. For example, Weisgraber et al. has shown that cysteine can be substituted for arginine at position 173 in a mutant ApoA1 termed ApoA1-Milano (Weisgraber et al. (1983) J. Biol. Chem. 258:2508-2513, herein incorporated by reference). ApoA1 polypeptide mimetics contemplated in the present invention can also include polypeptides from the ApoA1 forms and variants including, for example, apolipoprotein A-1 (Brewer et al., (1978)), apolipoprotein A-1 Milano (Weisgraber (1983)), apolipoprotein A-1 Paris (Bielicki and Oda (2002) Biochemistry 41:2089-2096), proapolipoprotein A-1, or any other mutant form of ApoA1 known in the art whether synthetically formed or naturally occurring.

Alternatively, the ApoA1 mimetics of the present invention can include an amphipathic helical peptides that closely mimic the class A amphipathic helix of human or mouse ApoA1 peptide (i.e., mimetics of ApoA1). The term “an amphipathic helical peptide” refers to a peptide comprising at least on amphipathic helix (amphipathic helical domain). Certain amphipathic helical peptides of this invention can comprise two or more (e.g., 3, 4, 5, etc.) amphipathic helices.

The term “class A amphipathic helix” refers to a protein structure that forms in an a-helix producing a segregation of a polar and nonpolar faces with the positively charged residues residing at the polar-nonpolar interface and the negatively charged residues residing at the center of the polar face (see, e.g., Segrest et al. (1990) Proteins: Structure, Function, and Genetics 8:103-117). Particularly preferred peptides may include greater than about 50% amino acid sequence identity with the polypeptide encoded by the exon encoding a class A amphipathic helix of human or mouse ApoA1. The peptide may be combined with a pharmacologically acceptable excipient (e.g., an excipient suitable for oral administration to a mammal). In one embodiment, the ApoA1 mimetic can be resistant to oxidation when administered to a subject. By “resistant to oxidation” or “oxidant resistant” as used in the specification and the claims, it is meant the ApoA1 mimetic according to the present invention has an amino acid sequence that is substantially similar to the amino acid sequence of ApoA1, ApoA1 fragments, or known mimetics of ApoA1 that contain at least on tryptophan and were at least on tryptophan residues is substituted with oxidant resistant residues, such as an oxidant resistant peptide residue, and for which ApoA1 lipid binding an efflux activities are retained.

In another embodiment, the oxidant resistant residue of the ApoA1 mimetic can include an aromatic peptide residue, such as phenylalanine. Examples of oxidant resistant ApoA1 mimetics are disclosed in U.S. patent application Ser. No. 12/256,822, filed Oct. 23, 2008, which is incorporated herein by reference in its entirety. For example, the oxidant resistant ApoA1 mimetic can include forms of human ApoA1, such as 4WF (tryptophans [W] at amino acids 8, 50, 72 and 108 mutated to phenylalanines [F]).

In another embodiment, the oxidant resistant ApoA1 mimetic used to treat cancer in a subject can fail to activate a proinflammatory cellular reaction including activation of nuclear factor kappa B transcription factor, or vascular cell adhesion molecule (VCAM) expression. For example, it was found that oxidant resistant forms of human ApoA1 that include 166 tyrosine (y) to glutamic acid (E) or aspartic acid (D) mutations retain Lecithin-cholesterol acyltransferase (LCAT) binding and are capable of stimulation LCAT activity as well as fail to activate a proinflammatory cellular reaction including activation of nuclear factor kappa B transcription factor, or vascular cell adhesion molecule (VCAM) expression.

In some embodiments of the present invention, the oxidant resistant ApoA1 can include the 4WF mutations in combination with Y166 E or D mutations and be administered to a subject to protect against a wide variety of cancers including, malignant melanoma, lung, breast, colon, brain, kidney, bladder, prostate, pancreatic, oral, head and neck, sarcomas, lymphomas (B-cell and T-cell) and adrenal cancer development as well as slow development of tumors, slow tumor metastasis, and lengthen survival times of the subject.

In certain embodiments of the invention, the ApoA1 mimetic have the following amino acid sequence:

(SEQ ID NO: 1)

DEPPQSPXDR VKDLATVYVD VLKDSGRDYV SQFEGSALGK
QLNLKLLDNX DSVTSTFSKL REQLGPVTQE FXDNLEKETE
GLRQEMSKDL EEVKAKVQPY LDDFQKKXQE EMELYRQKVE
PLRAELQEGA RQKLHELQEK LSPLGEEMRD RARAHVDALR
THLAPYSDEL RQRLAARLEA LKENGGARLA EYHAKATEHL
STLSEKAKPA LEDLRQGLLP VLESFKVSFL SALEEYTKKL NTQ

wherein X is either a tryptophan residue or an oxidant resistant residue (e.g., phenylalanine) and at least one of the four X's is an oxidant resistant residue. In other examples, at least two of the Xs of SEQ ID NO: 1 are an oxidant resistant residue, at least three of the Xs of SEQ ID NO: 1 are oxidant resistant residues, or all four of the Xs are oxidant resistant residues.

In certain embodiments, the ApoA1 mimetics, besides including tryptophan substituted wild-type or native forms of ApoA 1, can also include tryptophan substituted natural variants of ApoA1 that are known in the art. For example, Weisgraber et al. has shown that cysteine can be substituted for arginine at position 173 in a mutant ApoA1 termed ApoA1-Milano (Weisgraber et al. (1983) J. Biol. Chem. 258: 2508-2513, herein incorporated by reference). An ApoA1 mimetic based on ApoA1-Milano can therefore include the amino sequence of SEQ ID NO: 2.

(SEQ ID NO: 2)

MKAAVLTLAV LFLTGSQARH FXQQDEPPQS PXDRVKDLAT
VYVDVLKDSG RDYVSQFEGS ALGKQLNLKL LDNXDSVTST
FSKLREQLGP VTQEFXDNLE KETEGLRQEM SKDLEEVKAK
VQPYLDDFQK KXQEEMELYR QKVEPLRAEL QEGARQKLHE
LQEKLSPLGE EMRDRARAHV DALRTHLAPY SDELRQRLAA
RLEALKENGG ARLAEYHAKA TEHLSTLSEK AKPALEDLRQ
GLLPVLESFK VSFLSALEEY TKKLNTQ

wherein X is a tryptophan or an oxidant resistant residue (e.g., phenylalanine) and at least one X is substituted for an oxidant resistant residue.

In another embodiments of ApoA1 mimetic according to the present in invention is based on a known full-length mimetic of human ApoA1 peptide possessing a cysteine residue at position 151 of the mature ApoA1 (corresponding to position 175 in the sequence SEQ ID NO: 3). The ApoA1 mimetic in accordance with this example can include the amino acid sequence of SEQ ID NO: 3.

(SEQ ID NO: 3)

MKAAVLTLAV LFLTGSQARH FXQQDEPPQS PXDRVKDLAT
VYVDVLKDSG RDYVSQFEGS ALGKQLNLKL LDNXDSVTST
FSKLREQLGP VTQEFXDNLE KETEGLRQEM SKDLEEVKAK
VQPYLDDFQK KXQEEMELYR QKVEPLRAEL QEGARQKLHE
LQEKLSPLGE EMRDCARAHV DALRTHLAPY SDELRQRLAA
RLEALKENGG ARLAEYHAKA TEHLSTLSEK AKPALEDLRQ
GLLPVLESFK VSFLSALEEY TKKLNTQ;

wherein X is a tryptophan or an oxidant resistant residue (e.g., phenylalanine) and at least one X is substituted for an oxidant resistant residue (e.g., phenylalanine)

In some embodiments, the ApoA1 polypeptide mimetics contemplated in the present invention may include modified polypeptides from the ApoA1 forms and variants including, for example, apolipoprotein A-1 (Brewer et al., (1978)), apolipoprotein A-1 Milano (Weisgraber,(1983)), apolipoprotein A-1 Marburg, (Utermann et al., (1982) J. Biol. Chem. 257: 501-507), apolipoprotein A-1 Paris (Bielicki and Oda (2002) Biochemistry 41, 2089-2096), proapolipoprotein A-1, or any other mutant form of ApoA 1 known in the art whether synthetically formed or naturally occurring.

In other embodiments, the ApoA1 mimetics of the present invention can include an amphipathic helical peptides that closely mimic the class A amphipathic helix of human or mouse ApoA1 peptide (i.e., mimetics of ApoA1), wherein residues denoted by X can include a tryptophan residue or an oxidant resistant amino acid residue and at least one X is an oxidant resistant residue. The term “an amphipathic helical peptide” refers to a peptide comprising at least one amphipathic helix (amphipathic helical domain). Certain amphipathic helical peptides of this invention can comprise two or more (e.g., 3, 4, 5, etc.) amphipathic helices.

The term “class A amphipathic helix” refers to a protein structure that forms an a-helix producing a segregation of a polar and nonpolar faces with the positively charged residues residing at the polar-nonpolar interface and the negatively charged residues residing at the center of the polar face (see, e.g., Segrest et al. (1990) Proteins: Structure, Function, and Genetics 8: 103-117). Particularl peptides may include greater than about 50% amino acid sequence identity with the polypeptide encoded by the exon encoding a class A amphipathic helix of human or mouse ApoA1. The peptide may be combined with a pharmacologically acceptable excipient (e.g. an excipient suitable for oral administration to a mammal).

In certain embodiments, the ApoA1 mimetic is a fragment or mimetic of ApoA1, which is capable of promoting cholesterol efflux, and comprises one or more of the following amino acid sequences:

(SEQ ID NO: 4)

D-X-L-K-A-F-Y-D-K-V-A-E-K-L-K-E-A-F-,

(SEQ ID NO: 5)

D-X-F-K-A-F-Y-D-K-V-A-E-K-L-K-E-A-F-,

(SEQ ID NO: 6)

D-X-L-K-A-F-Y-D-K-V-A-E-K-F-K-E-A-F-,

(SEQ ID NO: 7)

D-X-F-K-A-F-Y-D-K-V-A-E-K-F-K-E-A-F-,

(SEQ ID NO: 8)

D-X-L-K-A-F-Y-D-K-V-F-E-K-F-K-E-F-F-,

(SEQ ID NO: 9)

D-X-L-K-A-F-Y-D-K-F-F-E-K-F-K-E-F-F-,

(SEQ ID NO: 10)

D-X-F-K-A-F-Y-D-K-F-F-E K-F-K-E-F-F-,

(SEQ ID NO: 11)

D-X-L-K-A-F-Y-D-K-V-F-E-K-F-K-E-A-F-,

(SEQ ID NO: 12)

D-X-L-K-A-F-Y-D-K-V-F-E-K-F-K-E-A-F-,

(SEQ ID NO: 13)

D-X-L-K-A-F-Y-D-K-V-F-E-K-L-K-E-F-F-,

(SEQ ID NO: 14)

D-X-L-K-A-F-Y-D-K-V-A-E-K-F-K-E-F-F-,

(SEQ ID NO: 15)

D-X-L-K-A-F-Y-D-K-V-F-E-K-F-K-E-F-F-,

(SEQ ID NO: 16)

E-X-L-K-L-F-Y-E-K-V-L-E-K-F-K-E-A-F-,

(SEQ ID NO: 17)

E-X-L-K-A-F-Y-D-K-V-A-E-K-F-K-E-A-F-,

(SEQ ID NO: 18)

E-X-L-K-A-F-Y-D-K-V-A-E-K-L-K-E-F-F-,

(SEQ ID NO: 19)

E-X-L-K-A-F-Y-D-K-V-F-E-K-F-K-E-A-F-,

(SEQ ID NO: 20)

E-X-L-K-A-F-Y-D-K-V-F-E-K-L-K-E-F-F-,

(SEQ ID NO: 21)

E-X-L-K-A-F-Y-D-K-V-A-E-K-F-K-E-F-F-,

(SEQ ID NO: 22)

E-X-L K-A-F-Y-D-K-V-F-E-K-F-K-E-F-F-,

(SEQ ID NO: 23)

D-X-L-K-A-L-Y-D-K-V-A-E-K-L-K-E-A-L-,

(SEQ ID NO: 24)

D-X-F-K-A-F-Y-E-K-V-A-E-K-L-K-E-F-F-,

(SEQ ID NO: 25)

D-X-F-K-A-F-Y-E-K-F-F-E-K-F-K-E-F-F-,

(SEQ ID NO: 26)

E-X-L-K-A-L-Y-E-K-V-A-E-K-L-K-E-A-L-,

(SEQ ID NO: 27)

E-X-L-K-A-F-Y-E-K-V-A-E-K-L-K-E-A-F-,

(SEQ ID NO: 28)

E-X-F-K-A-F-Y-E-K-V-A-E-K-L-K-E-F-F-,

(SEQ ID NO: 29)

E-X-L-K-A-F-Y-E-K-V-F-E-K-F-K-E-F-F-,

(SEQ ID NO: 30)

E-X-L-K-A-F-Y-E-K-F-F-E-K-F-K-E-F-F-,

(SEQ ID NO: 31)

E-X-F-K-A-F-Y-E-K-F-F-E-K-F-K-E-F-F-,

(SEQ ID NO: 32)

D-F-L-K-A-X-Y-D-K-V-A-E-K-L-K-E-A-X-,

(SEQ ID NO: 33)

E-F-L-K-A X-Y-E-K-V-A-E-K-L-K-E-A-X-,

(SEQ ID NO: 34)

D-F-X-K-A-X-Y-D-K-V-A-E-K-L-K-E-X-X-,

(SEQ ID NO: 35)

E-F-X-K-A-X-Y-E-K-V-A-E-K-L-K-E-X-X-,

(SEQ ID NO: 36)

D-K-L-K-A-F-Y-D-K-V-F-E-X-A-K-E-A-F-,

(SEQ ID NO: 37)

D-K-X-K-A-V-Y-D-K-F-A-E-A-F-K-E-F-L-,

(SEQ ID NO: 38)

E-K-L-K-A-F-Y-E-K-V-F-E-X-A-K-E-A-F-,

(SEQ ID NO: 39)

E-K-X-K-A-V-Y-E-K-F-A-E-A-F-K-E-F-L-,

(SEQ ID NO: 40)

D-X-L-K-A-F-V-D-K-F-A-E-K-F-K-E-A-Y-,

(SEQ ID NO: 41)

E-K-X-K-A-V-Y-E-K-F-A-E-A-F-K-E-F-L-,

(SEQ ID NO: 42)

D-X-L-K-A-F-V-Y-D-K-V-F-K-L-K-E-F-F-,

(SEQ ID NO: 43)

E-X-L-K-A-F-V-Y-E-K-V-F-K-L-K-E-F-F-,

(SEQ ID NO: 44)

D-X-L-R-A-F-Y-D-K-V-A-E-K-L-K-E-A-F-,

(SEQ ID NO: 45)

E-X-L-R-A-F-Y-E-K-V-A-E-K-L-K-E-A-F-,

(SEQ ID NO: 46)

D-X-L-K-A-F-Y-D-R-V-A-E-K-L-K-E-A-F-,

(SEQ ID NO: 47)

E-X-L-K-A-F-Y-E-R-V-A-E-K-L-K-E-A-F-,

(SEQ ID NO: 48)

D-X-L-K-A-F-Y-D-K-V-A-E-R-L-K-E-A-F-,

(SEQ ID NO: 49)

E-X-L-K-A-F-Y-E-K-V-A-E-R-L-K-E-A-F-,

(SEQ ID NO: 50)

D-X-L-K-A-F-Y-D-K-V-A-E-K-L-R-E-A-F-,

(SEQ ID NO: 51)

E-X-L-K-A-F-Y-E-K-V-A-E-K-L-R-E-A-F-,

(SEQ ID NO: 52)

D-X-L-K-A-F-Y-D-R-V-A-E-R-L-K-E-A-F-,

(SEQ ID NO: 53)

E-X-L-K-A-F-Y-E-R-V-A-E-R-L-K-E-A-F-,

(SEQ ID NO: 54)

D-X-L-R-A-F-Y-D-K-V-A-E-K-L-R-E-A-F-,

(SEQ ID NO: 55)

E-X-L-R-A-F-Y-E-K-V-A-E-K-L-R-E-A-F-,

(SEQ ID NO: 56)

D-X-L-R-A-F-Y-D-R-V-A-E-K-L-K-E-A-F-,

(SEQ ID NO: 57)

X-L-R-A-F-Y-E-R-V-A-E-K-L-K-E-A-F-,

(SEQ ID NO: 58)

D-X-L-K-A-F-Y-D-K-V-A-E-R-L-R-E-A-F-,

(SEQ ID NO: 59)

E-X-L-K-A-F-Y-E-K-V-A-E-R-L-R-E-A-F-,

(SEQ ID NO: 60)

D-X-L-R-A-F-Y-D-K-V-A-E-R-L-K-E-A-F-,

(SEQ ID NO: 61)

E-X-L-R-A-F-Y-E-K-V-A-E-R-L-K-E-A-F-,

(SEQ ID NO: 62)

D-X-L-K-A-F-Y-D-K-V-A-E-K-L-K-E-A-F-P-D-X-L-K-A-F-
Y-D-K-V-A-E-K-L-K-E-A-F,

(SEQ ID NO: 63)

D-X-L-K-A-F-Y-D-K-V-A-E-K-L-K-E-F-F-P-D-X-L-K-A-F-
Y-D-K-V-A-E-K-L-K-E-F-F,

(SEQ ID NO: 64)

D-X-F-K-A-F-Y-D-K-V-A-E-K-L-K-E-A-F-P-D-X-F-K-A-F-
Y-D-K-V-A-E-K-L-K-E-A-F,

(SEQ ID NO: 65)

D-K-L-K-A-F-Y-D-K-V-F-E-X-A-K-E-A-F-P-D-K-L-K-A-F-
Y-D-K-V-F-E-X-L-K-E-A-F,

(SEQ ID NO: 66)

D-K-X-K-A-V-Y-D-K-F-A-E-A-F-K-E-F-L-P-D-K-X-K-A-V-
Y-D-K-F-A-E-A-F-K-E-F-L,

(SEQ ID NO: 67)

D-X-F-K-A-F-Y-D-K-V-A-E-K-F-K-E-A-F-P-D-X-F-K-A-F-
Y-D-K-V-A-E-K-F-K-E-A-F,

(SEQ ID NO: 68)

D-X-L-K-A-F-V-Y-D-K-V-F-K-L-K-E-F-F-P-D-X-L-K-A-F-
V-Y-D-K-V-F-K-L-K-E-F-F,
and

(SEQ ID NO: 69)

D-X-L-K-A-F-Y-D-K-F-A-E-K-F-K-E-F-F-P-D-X-L-K-A-F-
Y-D-K-F-A-E-K-F-K-E-F-F;

wherein X is a tryptophan or an oxidant resistant residue (e.g., phenylalanine) and at least one X in each sequence is substituted for an oxidant resistant residue.

It will be appreciated that biologically functional equivalents, or even improvements, of the HDL, ApoA1, ApoA1 mimetics can be made, generally using ApoA1 as a starting point. Modifications and changes may be made in the structure of such a protein and still obtain a molecule having like or otherwise desirable characteristics. For example, certain amino acids may be substituted for other amino acids in the protein structure without appreciable loss of activity.

It is also well understood by the skilled artisan that, inherent in the definition of a “biologically functional equivalent” protein or polypeptide, is the concept that there is a limit to the number of changes that may be made within a defined portion of the molecule and still result in a molecule with an acceptable level of equivalent biological activity. Biologically functional equivalent proteins and peptides are thus defined herein as those proteins and peptides in which certain, not most or all, of the amino acids may be substituted. Of course, a plurality of distinct proteins/peptides with different substitutions may easily be made and used in accordance with the invention.

Amino acid substitutions are generally based on the relative similarity of the amino acid side-chain substituents, for example, their hydrophobicity, hydrophilicity, charge, size, and the like. An analysis of the size, shape and type of amino acid side-chain substituents reveals that arginine, lysine, and histidine are all positively charged residues; that alanine, glycine and serine are all a similar size. Therefore, based upon these considerations, arginine, lysine and histidine; alanine, glycine and serine are defined herein as biologically functional equivalents. Following the procedures noted in the published application by Alton et al. (WO83/04053), one can readily design and manufacture genes coding for microbial expression of polypeptides having primary conformations which differ from that herein specified in terms of the identity or location of one or more residues (e.g. substitutions, terminal and intermediate additions and deletions). Alternately, modifications of cDNA and genomic genes may be readily accomplished by well-known site-directed mutagenesis techniques and employed to generate analogs and derivatives of ApoA1. Such products would share at least one of the biological properties of ApoA1 but may differ in others.

The HDL, ApoA1, and/or ApoA1 mimetics of the present invention may be purified and isolated. The term “purified and isolated” herein means substantially free of unwanted substances so that the present lipoproteins can be used to treat cancer. For example, one may have a modified recombinant human ApoA1 mimetic polypeptide substantially free of other human proteins or pathological agents. These polypeptides are also characterized by being a product of mammalian cells, or the product of chemical synthetic procedures or of prokaryotic or eukaryotic host expression (e.g., by bacterial, yeast, higher plant, insect and mammalian cells in culture) of exogenous DNA sequences obtained by genomic or cDNA cloning or by gene synthesis. The products of expression in typical yeast (e.g., Saccharomyces cerevisiae) or prokaryote (e.g., E. coli) host cells are free of association with any mammalian proteins. The products of expression in vertebrate (e.g., non-human mammalian (e.g., COS or CHO) and avian) cells are free of association with any human proteins. Depending upon the host employed, and other factors, polypeptides of the invention may be glycosylated with mammalian or other eucaryotic carbohydrates or may be non-glycosylated. Polypeptides of the invention may also include an initial methionine amino acid residue (at position-1 with respect to the first amino acid residue of the polypeptide).

The lipoproteins and peptides of the invention can be purified by art-known techniques such as reverse phase chromatography high performance liquid chromatography, ion exchange chromatography, gel electrophoresis, affinity chromatography and the like. The actual conditions used to purify a particular peptide will depend, in part, on the synthesis strategy and on factors, such as net charge, hydrophobicity, hydrophilicity, etc., and will be apparent to those having skill in the art. Multimeric branched peptides can be purified, e.g., by ion exchange or size exclusion chromatography.

For affinity chromatography purification, any antibody which specifically binds the peptide may be used. For the production of antibodies, various host animals, including but not limited to rabbits, mice, rats, etc., may be immunized by injection with a peptide. The peptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as hemocyanin, dinitrophenol, and potentially useful human adjuvants such as BCG (bacilli Calmette-Guerin) and Corynebacterium parvum.

### III. Cancers

Cancers and related disorders that can be prevented, treated, or managed by methods and compositions of the present invention include but are not limited to the following cancers: leukemias, such as but not limited to, acute leukemia, acute myeloid leukemia (AML), chronic myelogenous (or myeloid) leukemia (CML), acute lymphocytic leukemia, acute myelocytic leukemias, such as, myeloblastic, promyelocytic, myclomonocytic, monocytic, and erythroleukemia leukemias and myelodysplastic syndrome; chronic leukemias, such as but not limited to, chronic myelocytic (granulocytic) leukemia, chronic lympocytic leukemia, hairy cell leukemia, polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochrmocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but not limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to squamous cell carcinoma, and adenocarcinoma; uterine cancers such as but not limited to endometrial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcionomas such as but not limited to pappilary, nodular, and diffuse; lung cancers such as non-small cell lung cancer, squamous cell carcinoma (epidermoid carcinoma), adenocarcinoma, large-cell carcinoma, and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenocystic carcinoma; pharynx cancers such as but not limited to squamous cell cancer, and verrucous; skin cancers such as but not limited to basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxsarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America)

In some embodiments, the compositions and methods of the present invention are useful in the treatment or prevention of a variety of cancers or other abnormal proliferative diseases, including (but not limited to) the following: carcinoma, including that of the bladder, breast, prostate, rectal, colon, kidney, liver, lung, ovary, pancreas, stomach, cervix, thyroid and skin; including squamous cell carcinoma; hematopoictic tumors of myeloid lineage, including acute and chronic myelogenous leukemias and promoclocytic leukemia; tumors of mesenchymal origin; including fibrosarcoma and rhabdomyoscarcoma; other tumors, including melanoma, seminoma, tetratocarcinoma, neuroblastoma, and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, hormone dependent tumors of the breast, prostate and ovary, and precancerous lesions such as familial adenomatous polyposis, and myelodysplastic syndromes. In specific embodiments, malignancy or dysproliferative changes (such as metaplasis and dyplasias), or hyperproliferative disorders, are treated in the skin, lung, colon, rectum, breast, prostate, bladder, kidney, pancreas, ovary, or uterus. In other specific embodiments, sarcoma, melanoma, small lung carcinoma, or leukemia is treated.

In some embodiments, the cancer is malignant. In other embodiments, the disorder to be treated is a pre-cancerous condition. In a specific embodiment, the pre-cancerous condition is high-grade prostatic intraepithelial neoplasia (PIN), fibroadenoma of the breast, or fibrocystic disease.

In certain embodiments, the compositions of the invention can be delivered to cancer cells by site-specific means. Cell-type specific delivery can be provided by conjugating a therapeutic agent to a targeting molecule, for example, one that selectively binds to the affected cells. Methods for targeting include conjugates, such as those described in U.S. Pat. No. 5,391,723. Targeting vehicles, such as liposomes, can be used to deliver a compound, for example, by encapsulating the compound in a liposome containing a cell-specific targeting molecule. Methods for targeted delivery of compounds to particular cell types are well-known to those skilled in the art.

### IV. Pharmaceutical Compositions

The pharmaceutical composition can include a pharmaceutically acceptable carrier and a non-toxic therapeutically effective amount of the compositions of the present invention.

The phrases “pharmaceutically or pharmacologically acceptable” refer to molecular entities and compositions that do not produce an adverse, allergic, or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial, and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

Examples of carriers include solvents and dispersion media containing, for example, water, ethanol, polyol (for example, glycerol, propylene glycol, and liquid polyethylene glycol, and the like), mixtures thereof, and vegetable oils. In many cases, it will be preferable to include isotonic agents, for example, by the use of a coating, such as lecithin, by the maintenance of the required particle size in the case of dispersion and/or by the use of surfactants.

The present invention contemplates the administration of the described pharmaceutical compositions by various routes. Pharmaceutical compositions comprising the compositions of the present invention may be administered by any route that ensures bioavailability in the circulation. These routes can include, but are by no means limited to parenteral administration, systemic administration, oral administration, nasal administration, rectal administration, intraperitoneal injection, intravascular injection, subcutaneous injection, transcutaneous administration, inhalation administration, and intramuscular injection.

Injectable preparations include sterile suspensions, solutions or emulsions of the active ingredient in aqueous or oily vehicles. The compositions may also contain formulating agents, such as suspending, stabilizing and/or dispersing agent. The formulations for injection may be presented in unit dosage form, e.g. in ampoules or in multidose containers, and may contain added preservatives.

Alternatively, the injectable formulation may be provided in powder form for reconstitution with a vehicle, including but not limited to sterile pyrogen free water, buffer, dextrose solution, etc., before use. To this end the compositions of the present invention may be lyophilized, or the co-lyophilized peptide-lipid complex may be prepared. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection. Thus, for example, the active ingredient may be formulated with polymeric or hydrophobic materials (e.g., as an emulsion in an acceptable oil) or ion exchange resins, or as sparingly soluble derivatives.

Alternatively, transdermal delivery systems manufactured as an adhesive disc or patch which slowly releases the active ingredient for percutaneous absorption may be used. To this end, permeation enhancers may be used to facilitate transdermal penetration of the active ingredient.

For oral administration, the pharmaceutical compositions may take the form of, for example, tablets or capsules prepared by conventional means with pharmaceutically acceptable excipients, such as binding agents (e.g., pregelatinised maize starch, polyvinylpryrrolidone or hydroxypropyl methylcellulose); fillers (e.g., lactose, microcrystalline cellulose or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives, such as suspending agents (e.g., sorbitol syrup, cellulose derivatives or hydrogenated edible fats); emulsifying agents (e.g., lecithin or acacia); non-aqueous vehicles (e.g., almond oil, oily ester, ethyl alcohol or fractioned vegetable oils): and preservatives (e.g., methyl or propyl-p-hydroxybenzoates or ascorbic acid). The preparations may also contain buffer salts, flavoring, coloring, and sweetening agents as appropriate. Preparations for oral administration may be suitably formulated to give controlled release of the active compound.

For buccal administration, the compositions may take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a propellant, e.g., dichlorodifluoromethane, trichlorofluoromethane, dichlorotetrafluoroethane, carbon dioxide or other gas. In the case of a pressurized aerosol, the dosage unit may be determined by providing a valve to deliver a metered amount. Capsules and cartridges of e.g. gelatin for use in an inhaler or insufflators may be formulated containing a powder mix of the compound and a suitable powder base such as lactose or starch.

The compositions may, if desired, be presented in a pack or dispenser device, which may contain one or more unit of dosage forms containing the active ingredient. The pack may for example comprise metal or plastic foil, such as a blister pack. The pack or dispenser device may be accompanied by instructions for administration.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

Under ordinary conditions of storage and use, all such preparations should contain a preservative to prevent the growth of microorganisms. The prevention of the action of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate and gelatin.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Pharmaceutical “slow release” capsules or “sustained release” compositions or preparations may be used and are generally applicable. Slow release formulations are generally designed to give a constant drug level over an extended period and may be used to deliver ApoA1 mimetic polypeptides or fragments thereof in accordance with the present invention. In certain embodiments, liposomes and/or nanoparticles may also be employed with the HDL, ApoA1, and/or ApoA1 mimetics. The formation and use of liposomes is generally known to those of skill in the art, as summarized below. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core. HDL, ApoA1, and/or ApoA1 mimetics can also formulated be into phospholipid discs of between 8 and 20 nm, through spontaneous reaction with phospholipid liposomes, or through the cholate dialysis procedure.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

I claim:

1. A method of treating cancer in a subject comprising: administering to a subject:
a) a chemotherapeutic agent, wherein said chemotherapeutic agent induces at least some level of cardiotoxicity or cardiomyopathy in said subject when administered at a therapeutic level, and
b) a composition comprising a therapeutic agent and/or a nucleic acid sequence encoding said therapeutic agent, wherein said therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in said subject,
wherein said composition is effective in preventing or reducing the level of cardiotoxicity and/or cardiomyopathy induced by said chemotherapeutic agent, and
wherein said administering is effective to prevent, suppress, and/or inhibit cancer cell growth in said subject.

2. The method of claim 1, wherein said therapeutic agent comprises a high-density lipoprotein (HDL) or HDL mimetic.

3. The method of claim 1, wherein said therapeutic agent comprises preproapoliprotein (preproApoA1).

4. The method of claim 1, wherein said composition is injected and/or infused into said subject.

5. The method of claim 1, wherein said nucleic acid sequence comprises an ApoA1 mRNA sequence.

6. The method of claim 1, wherein said nucleic acid sequence comprises an ApoA1 mimetic mRNA sequence.

7. The method of claim 1, wherein said therapeutic agent comprises said induction agent.

8. The method of claim 7, wherein said induction agent comprises a small molecule compound.

9. The method of claim 7, wherein said induction agent comprises a nucleic acid sequence encoding the HNF-4 gene.

10. The method of claim 7, wherein said induction agent comprises a statin that is able to cause an increased expression of HDL or ABCA1.

11. The method of claim 7, wherein said induction agent is niacin.

12. The method of claim 7, wherein said induction agent is a fibrate that is able to cause increased expression of HDL.

13. The method of claim 1, wherein said therapeutic agent comprises said binding agent specific for oxidized ApoA1.

14. The method of claim 13, wherein said binding agent comprises an antibody or antigen-binging portion thereof.

15. The method of claim 1, wherein said chemotherapeutic agent is administered with said composition to said subject at a dosage level that causes about the same level of cardiotoxicity and/or cardiomyopathy induced when said chemotherapeutic agent is administered without said composition to said subject.

16. A pharmaceutical composition for treating cancer in a subject comprising:
a) a chemotherapeutic agent, wherein said chemotherapeutic agent induces at least some level of cardiotoxicity or cardiomyopathy in said subject when administered at a therapeutic level, and
b) a therapeutically effective amount of a therapeutic agent and/or a nucleic acid sequence encoding said therapeutic agent, wherein said therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in said subject.

17. The pharmaceutical composition of claim 16, wherein said therapeutic agent comprises a high-density lipoprotein.

18. The pharmaceutical composition of claim 16, wherein said therapeutic agent comprises preproapoliprotein (preproApoA1).

19. The pharmaceutical composition of claim 16, wherein said nucleic acid sequence comprises an ApoA1 mRNA sequence.

20. A method of treating cardiotoxicity or cardiomyopathy comprising: administering, to a subject with cardiotoxicity or cardiomyopathy, a composition comprising a therapeutic agent and/or a nucleic acid sequence encoding said therapeutic agent, wherein said therapeutic agent comprises: i) apolipoprotein A-1 (ApoA1), ii) an ApoA1 mimetic, iii) a binding agent specific for oxidized ApoA1, and/or iv) an induction agent that induces increased endogenous expression of apolipoprotein A-1 (ApoA1) in said subject,
wherein said administering is effective in preventing or reducing the level of cardiotoxicity and/or cardiomyopathy in said subject.

21. The method of claim 20, wherein said cardiotoxicity or cardiomyopathy is induced by a chemotherapeutic agent previously taken by said subject.
